(19) United States
(12) Patent Application Publication
(10) Pub. No.: US 2017/0354526 A1
(43) Pub. Date: Dec. 14, 2017

(54) An Orthotic for Muscle Imbalance and Posture Correction and Lumbopelvic Support

(71) Applicant: Jim Li, Gilberton (AU)

(72) Inventor: Jim Li, Gilberton (AU)

(21) Appl. No.: 15/544,633

(22) PCT Filed: Jan. 19, 2016
(86) PCT No.: PCT/AU2016/050020

§ 371 Date: Jul. 19, 2017

(30) Foreign Application Priority Data
Jan. 19, 2015 (AU) 2015900141

(51) Int. Cl. A61F 5/01; A63B 23/04; A63B 21/055; A61H 1/00; A63B 21/00; A61F 5/02

(52) U.S. Cl. CPC A61F 5/0104; A63B 21/4011; A61F 5/028; A63B 21/0552; A61H 1/008; A63B 21/4025; A63B 21/4009; A63B 23/0482; A63B 2209/14

(57) ABSTRACT

There is provided an orthotic configured for muscle imbalance correction. The orthotic has a strapping configured for physically activating an undercompensating muscle group by physically pulling substantially in-line with muscle fibres of the undercompensating muscle group to assist muscle activation and physically deactivating an overcompensating muscle group by physically pulling and therefore compressing across muscle fibres of the overcompensating muscle group to provide muscle resistance. Examples of the undercompensating muscle group are at least one of the gluteus maximus, medius, and minimus, vastus medialis and vastus medialis oblique. Examples of the overcompensating muscle group are at least one of the tensor fascia latae and iliopsoas.

### FIELD OF THE INVENTION

The present invention relates to orthotics and in particular, but not necessarily entirely, to an orthotic for muscle imbalance and posture correction and lumbopelvic support.

### BACKGROUND OF THE INVENTION AND SUMMARY OF THE INVENTION

There will be described herein and orthotic offering muscle imbalance and/or posture correction and/or lumbopelvic support utilising an entirely differing corrective mechanism as is utilised in the prior art (including that prior art which is described with reference to FIGS. 1, 2 and 3 herein for contradistinction) wherein, as will be appreciated, while the prior art generally utilises proprioception feedback for wearer initiated correction, the orthotic provided herein provides physical restraint including for the purposes of physically pulling in line with undercompensating muscle groups to activate these muscle groups and physically pulling across and so as to compress overcompensating muscle groups so as to deactivate these overcompensating muscle groups.

Now, lower back pain is very problematic, especially with today's sedentary society. It is estimated that 70-90% of the population will experience back pain at some point in their lives.

Low back pain may be acute or chronic. Chronic pain is characterised in lasting longer than 6 weeks. Acute low back may be recurrent and rarely triggered by a specific traumatic incident. Repeated episodes reveal persistent over compensation and dysfunction leading to chronic pain. It is estimated that 90% of all low back pain is classified as non-specific low back pain, ruling out specific conditions such as Disc herniation, nerve root impingement, intervertebral disc disease and Grade 2 and above spondylolisthesis and the like. As such, the cause for low back pain in mostly caused by a multitude of factors that lead to biomechanical overcompensation and dysfunction.

One such cause of biomechanical overcompensation and dysfunction is exacerbated by a sedentary lifestyle where in, for example, being seated for prolonged periods causes the weakening of the gluteal muscles.

Gluteus maximus/medius weakness leads to lower crossed/pelvic cross syndrome responsible for large percentage of causes for chronic low back pain and hip impingement and furthermore causes the internal rotation of the femur leading to various complications such as pubic dysfunction, hip impingement, ITB syndrome, patella maltracking, runner's knees and overpronation of the feet.

Muscles in the human body work together in kinetic chains/subsystems of muscles activation. Therefore, in order to make changes to posture, the entire kinetic chain of muscles needs to be influenced.

Existing orthotic arrangements have attempted to remedy low back pain. However, current orthotic braces are too rigid and therefore only usefully initially for pain relief but cannot be worn for prolonged periods because of the patient's increasing reliance on the additional support leading to the counterproductive wasting of core muscles. As such, existing orthotic braces for lower back pain are generally suited therefore for pain relief only but serve no or little rehabilitative advantages.

Furthermore, current research indicates that lumbopelvic stability is a major contributing factor to chronic low back pain. However, existing orthotic lumbar braces fail to address lumbopelvic stability.

Furthermore, existing orthotic pelvic braces configured for lumbopelvic stability become displaced from optimal positions in use and therefore are unreliable. Furthermore, these existing orthotic pelvic braces do not address the muscle imbalance issue.

Yet further, whereas compressive garments exist, such garments serve only in supporting recovery and cannot address biomechanical issues on account of their inability to provide active (as opposed to merely reactive) compression.

As such, the present embodiments seeks to provide an orthotic, which will overcome or substantially ameliorate at least some of the deficiencies of the prior art, or to at least provide an alternative.

Specifically, in embodiments, the orthotic provided herein is adapted to provide muscle imbalance correction through the activation of certain weakened muscles and the deactivation of certain other overcompensating muscles, the provision of posture correction through the external lateral hip rotation and the above muscle imbalance correction and the provision of lumbopelvic support through the compression of the pelvis, especially the ASIS and PSIS skeletal landmarks.

Now, for contradistinction with existing arrangements, reference is made to FIG. 1 of D1 (U.S. Pat. No. 9,050,179 B2) showing a prior art garment adapted to be worn by a wearer. The garment includes a main body portion that is configured to be worn over at least a portion of the wearer's lumbo-pelvic region, hips and at least a portion of the legs, wherein the main body portion includes first and second leg portions that each define an axis. The garment also includes a strap system that includes a plurality of leg straps releasably affixed to the main body portion. Each of the leg straps includes at least one connector for affixing the leg strap to the main body portion. Each of the leg straps overlies a grip layer that is adapted to contact the wearer's skin when the garment is worn.

However, the garment of FIG. 1 is adapted to apply pressure the user's skin in order to proprioceptively stimulate cutaneous nerve receptors. Specifically, garment creates a sensation/cue on the skin through the design that specifically bends, compresses and directs the nerve receptors in the skin (peripheral nervous system nerve endings/square inch on skin, in muscles, tendons, joint lining, etc.) to be pulled in such a way and in such a specific direction that tells the brain to instantly relax and lengthen specific, over used, under stretched muscles while the brain simultaneously commands the opposite (front to back and/or side to side), weak, under toned, under supportive muscles to contract, tone and support the wearer's core and lower extremities.

However, as will be appreciated from the ensuing description, the present embodiments described herein do not rely on proprioceptive input. Rather, the present embodiments exert physical forces to achieve the desired outcomes. Specifically, for the muscle imbalance correction, the orthotic according to the present embodiments is adapted to pull substantially in line with the muscle fibres of certain weakened muscle groups so as to enhance the activation of these weakened muscle groups and to compress, or pull across the muscle fibres of certain other overcompensating muscle groups so as to deactivate these muscle groups so as to favour the correction of the muscle imbalance.

Furthermore, the orthotic provided herein in accordance with the present embodiments as will be described in further detail below is adapted to provide forces to not only provide for the lateral rotation of the hip, but also for the extension of the hip so as to provide for posture correction. Yet furthermore, the orthotic according to the present embodiments is adapted to provide pressure about the pelvis, especially the ASIS and PSIS regions to enhance form closure for lumbopelvic support, especially being advantageous for pain reduction purposes and therefore the facilitation of subsequent exercise to provide posture and muscle correction. This combination brings about an external force strong enough to exhibit a change in the whole kinetic chain during movement, which differs to the garment of FIG. 1 which brings about activation through proprioceptively stimulating nerve receptors in individual muscles.

Next, referring to FIG. 2 showing D2 (U.S. Pat. No. 8,435,202 B2), there is shown a supportive orthotic device further in accordance with the prior art for supporting the hip, groin, and/or thigh of a user. The device has a waist region sized to extend around the waist of the user. The device also has a first surface and a second surface coupled to the waist region. A thigh strap has a first end extending from the first surface of the device and a second end selectively coupled to the second surface and wraps around the user's thigh in a fastened configuration. A groin strap has a first end extending from the first surface of the device and a second end selectively coupled to the second surface and wraps around the user's groin area in a fastened configuration.

The orthotic of a FIG. 2 is adapted to promote adduction of the hips and thighs of the user while resisting abduction of the hips and thighs.

However, the orthotic of FIG. 2 fails to provide the muscle imbalance correction properties of the present orthotic as will be described in further detail below in that the orthotic of FIG. 2 fails to favour the activation of certain weakened muscles and the deactivation of certain other overcompensating muscles. Furthermore, the orthotic of FIG. 2 fails to provide external rotational forces to the hip and counteractively adducts the hip not abduct. Furthermore, the orthotic of FIG. 2 fails to provide the lumbopelvic support provided by the orthotic of the present embodiments.

Yet further, FIG. 3 of D3 (US 20100088803 A1) shows a garment to be positioned about one or more body portions of a person performing a physical activity comprises a garment region having a garment region elasticity and a training region having a training region elasticity, the garment region stretching a greater distance as the result of an applied force than the training region under the same applied force. The training region at least partially defines a tensioning system of a biofeedback device. The biofeedback device is configured to provide sensory cues (e.g., feedback forces) to the person performing a physical activity when the person's biomechanic position is sub-optimal, the sensory cues indicating to the person that their biomechanic position is sub-optimal and indicating to the person how to adjust their one or more body portions towards a more optimal biomechanic position. The garment may provide for training and help the person achieve optimal biomechanic motion.

However, as alluded to above, whereas the orthotic of FIG. 3 utilises proprioceptive input to train the brain (much like the orthotic as provided in FIG. 1), the orthotic of the present embodiments actually provides physical forces to the musculoskeletal system of the patient to provide the muscle imbalance and posture correction and lumbopelvic support properties described herein.

For example, paragraph 205 of D3 describes the internal rotation of the wearer's hips wherein, the inward rotation of the wearer's hips creates tension in a “training region”. The tension applied to the training region allows for proprioceptive feedback to the wearer informing the wearer that the user should rotate their hips outward back to the optimal biomechanical position. As such, D3 does not actually describe the physical pulling in line with the muscle fibres to assist muscle activation as is provided herein. D3 further neither describes muscle deactivation by physically pulling across the muscle fibres of an overcompensating muscle group as is provided herein.

As such, with the foregoing in mind, in accordance with one aspect, there is provided an orthotic configured for muscle imbalance correction wherein the orthotic comprises strapping configured for: physically activating an undercompensating muscle group comprising at least one of the gluteus maximus, medius, and minimus, vastus medialis and vastus medialis oblique by physically pulling substantially in-line with muscle fibres of the undercompensating muscle group to assist muscle activation; and physically deactivating an overcompensating muscle group comprising at least one of the tensor fascia latae and iliopsoas by physically pulling and therefore compressing across muscle fibres of the overcompensating muscle group to provide muscle resistance.

The strapping may comprise a pair of straps configured for respectively travelling posteriorly from respective opposite superior hip regions across a pelvic region to respective opposite inferior hip regions so as to be configured for: physically pulling around the overcompensating muscle group at the superior and inferior hip regions so as to physically deactivate these muscles at these regions; and physically pulling in line with the undercompensating muscle group between the at the superior and inferior hip regions so as to physically activate these muscles.

The straps may be tension adjustable.

The orthotic may comprise ergonomically located lateral anterior strap tension adjustment means positioned to allow the wearer to self-adjust the tension of the straps.

The orthotic may be further configured for posture correction wherein the strapping may be further configured for physically exerting hip rotation forces for the lateral rotation of the hips to favour a substantially neutral hip position.

The pair of straps may be configured for travelling from the respective opposite inferior hip regions across respective anterior thigh surfaces to exert lateral rotational forces on each leg.

The pair of straps may each terminate at respective medial knee regions

The orthotic may comprise a frictional engagement to enhance the purchase of the straps at at least one of respective thigh surfaces and medial knee regions.

The strapping may be further configured for physically exerting hip extension forces for the extension of the hip to favour a substantially neutral pelvis tilt.

The orthotic may be further configured for lumbopelvic support wherein the strapping may be configured for physically exerting compressive forces for compressing the pelvis to enhance form closure.

The strapping may be configured for compressing the anterior superior iliac spine (ASIS) and posterior superior iliac spine (PSIS).

The orthotic may further comprise appropriately padding located substantially at at least one of the ASIS and PSIS regions to enhance compressive force exerted by the straps.

The orthotic may further comprise a compression garment.

The compression garment may comprise a sleeve for slideably engaging at least a portion of the straps.

It is to be understood that, if any prior art information is referred to herein, such reference does not constitute an admission that the information forms part of the common general knowledge in the art, in Australia or any other country.

Other aspects of the invention are also disclosed.

### BRIEF DESCRIPTION OF THE DRAWINGS

Notwithstanding any other forms which may fall within the scope of the present invention, a preferred embodiments of the disclosure will now be described, by way of example only, with reference to the accompanying drawings in which:

FIGS. 1-3 show various prior art arrangements;

FIG. 4 shows the musculoskeletal effects of weakened gluteus maximus muscles;

FIG. 5 shows a side view of a lower body orthotic in accordance with a preferred embodiment of the present disclosure;

FIG. 6 shows an interior view of the orthotic of FIG. 5 in accordance with the preferred embodiment of the present disclosure;

FIG. 7 shows a posterior view of the orthotic of FIG. 5 in accordance with the preferred embodiment of the present disclosure;

FIG. 8 shows a posterior view of an outline of the orthotic of FIG. 5 overlaid various muscle groups in accordance with the preferred embodiment of the present disclosure

FIG. 9 shows an anterior view of an outline of the orthotic of FIG. 5 overlaid various muscle groups in accordance with the preferred embodiment of the present disclosure;

FIG. 10 shows a posterior view of an upper body orthotic in accordance with an embodiment of the present disclosure; and

FIG. 11 shows an anterior view of an upper body orthotic in accordance with an embodiment of the present disclosure.

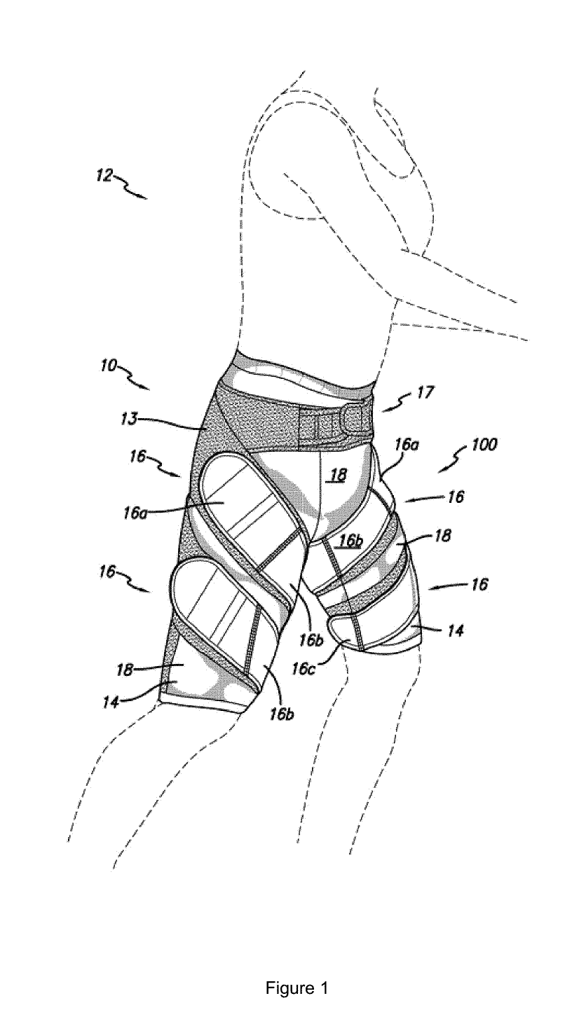

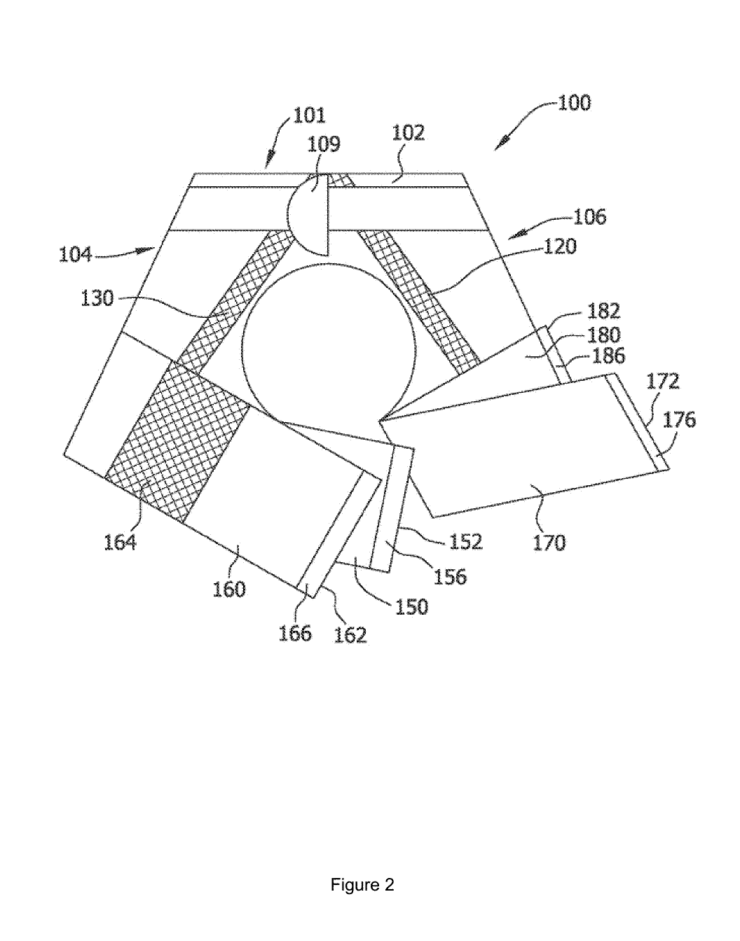

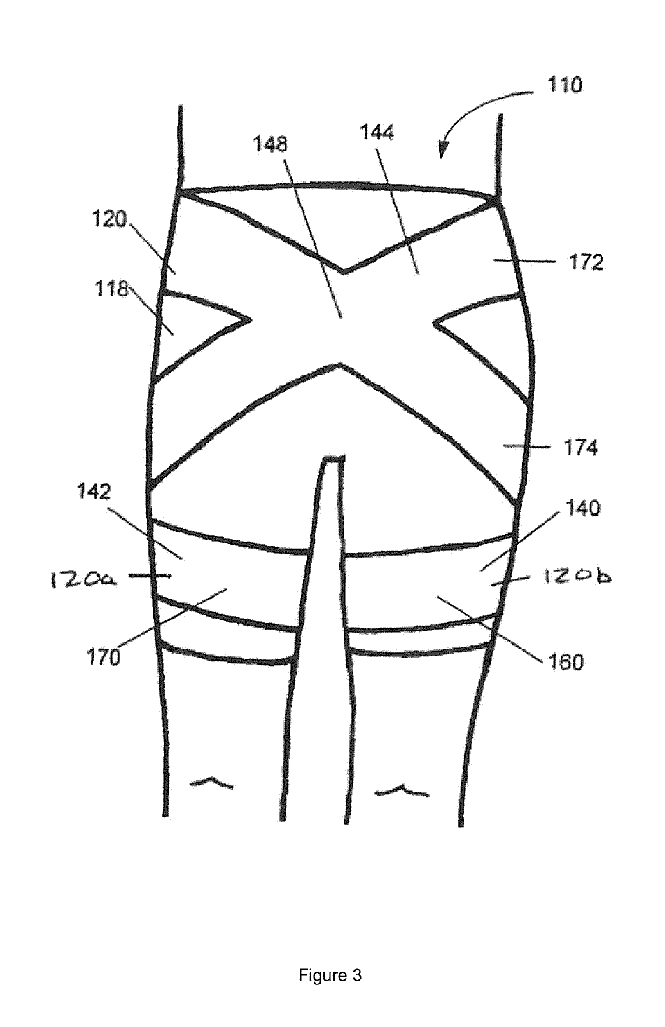

### DESCRIPTION OF EMBODIMENTS

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure.

Before the structures, systems and associated methods relating to the orthotic are disclosed and described, it is to be understood that this disclosure is not limited to the particular configurations and materials and the like disclosed herein as such may vary somewhat. It is also to be understood that the terminology employed herein is used for the purpose of describing particular embodiments only and is not intended to be limiting since the scope of the disclosure will be limited only by the claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterised by,” and grammatical equivalents thereof are inclusive or open-ended terms that do not exclude additional, unrecited elements or method steps.

It should be noted in the following description that like or the same reference numerals in different embodiments denote the same or similar features.

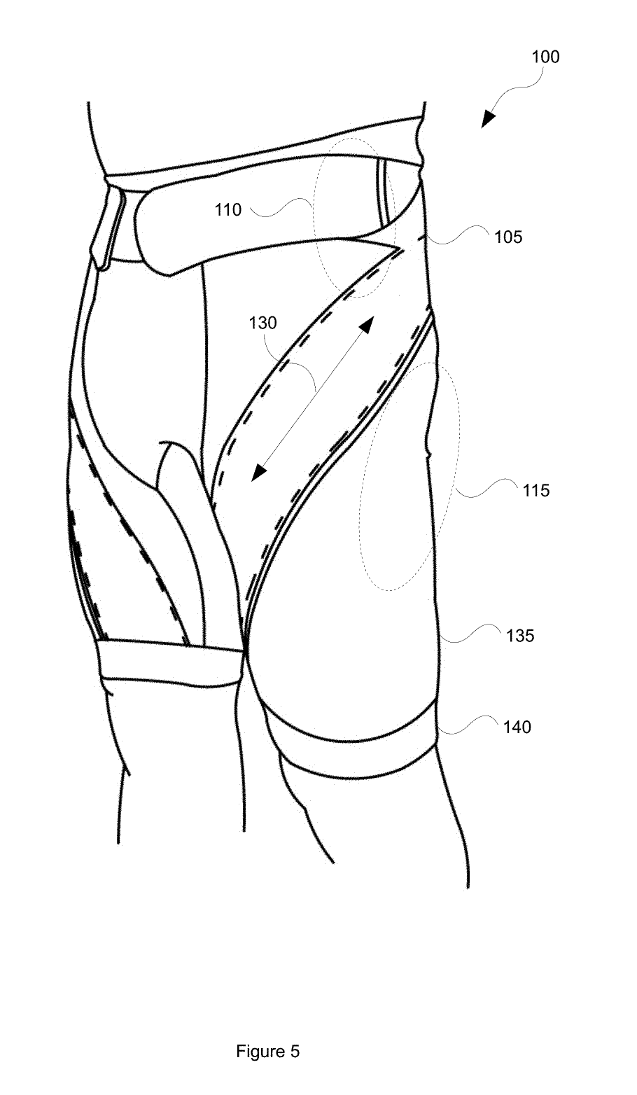

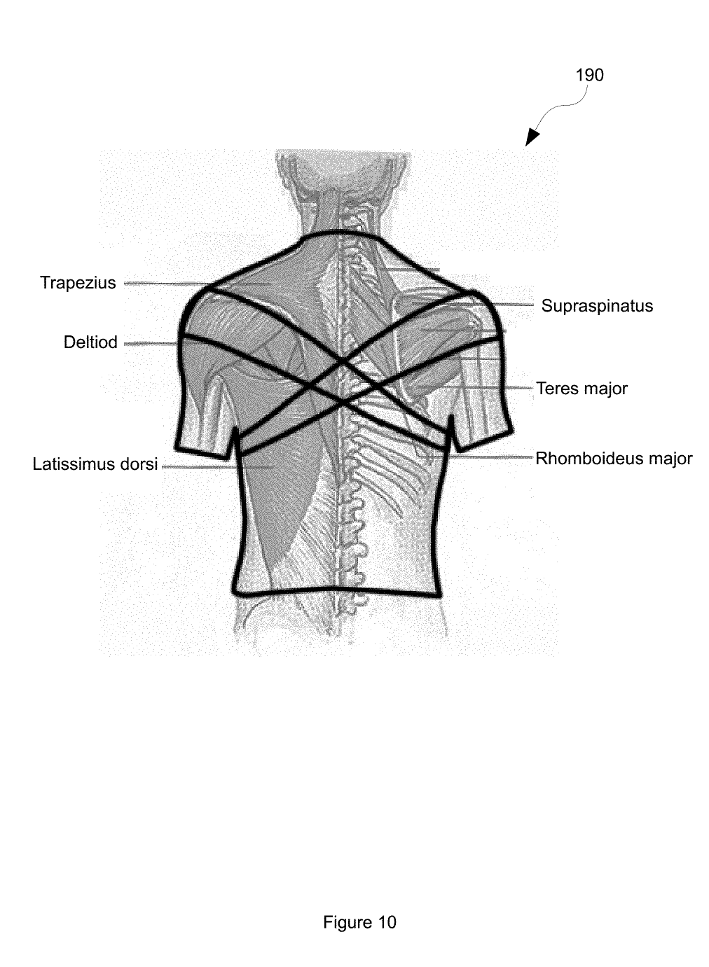

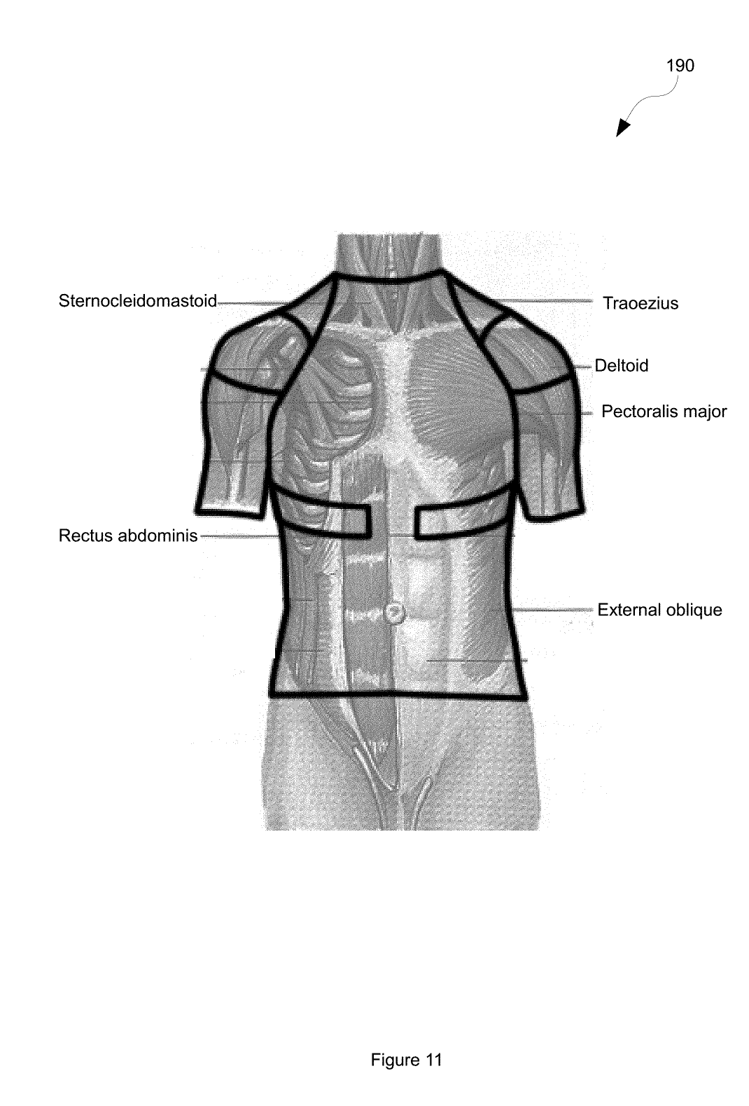

Turning now to FIGS. 5-9, there is shown a lower body orthotic 100. As alluded to above, in embodiments the orthotic 100 is adapted to provide muscle imbalance correction, posture correction and lumbopelvic support each of which is discussed in further detail below. Similarly, FIG. 10-11 shown and upper body orthotic 190.

### Muscle Imbalance Correction

There will now be described the technical features adapted to provide the orthotic 100 with muscle imbalance correction properties.

Now, by way of background, muscle imbalance occurs when opposing muscles shorten or lengthen in relation to each other. Such may be caused by specific training, or adopting certain positions for extended periods of time, such as being predominantly sedentary, for example.

As will be described in further detail below, the orthotic 100 primarily targets the gluteus maximus and medius muscles for hip stabilisation, these being the muscles most commonly weakened during muscle imbalance. By stabilising the hip, other weakened and/or inactive muscles along the kinematic chain will be activated, including the knee stabiliser such as the vastus medialis and vastus medialis oblique, for foot ankle stabilisers such as the tibialis posterior, the deep core stabilises of the trunk, for pubic and hip stabilizers posterior adductor magnus and the like.

The gluteus maximus is the strongest and biggest muscle in the body. It is well-known that the gluteus maximus serves not only hip extensor purposes but also pelvic and spinal stabilisation. In this regard, the gluteal muscles (comprising the gluteus maximus, gluteus medius and gluteus minimus) stabilise the hip by counteracting the gravity induced hip adduction torque and maintain proper leg alignment by eccentrically controlling adduction and internal rotation of the thigh.

As such, the gluteus maximus allows the maintaining of an upright position required for bipedalism. Through an evolutionary process, the gluteus maximus enlarged in humans as a means to stabilise the trunk while standing and counteract the high-impact forces that tend to flex the trunk anteriorly during running and sprinting. Consequently, the gluteal muscles gradually lose tone in a sedentary lifestyle, commonplace today.

In addition to sedentary lifestyle, prolonged sitting is far more commonplace—transportation, side sleeping, office work, eating all involve the same position. Our bodies proprioceptively adapt to common positions to make them easier for us. Thus the anterior muscles become shortened to allow easier activation and posterior muscles such as gluteal maximus and medius become lengthened reciprocally. This may progress to a point where it becomes very difficult to activate these muscles without shortening their muscle length first.

As result of weakened gluteus maximus muscles, a muscle imbalance occurs known as lower crossed syndrome wherein the anterior hip/thigh muscles become shorter and active while the posterior gluteal muscles become long and inactive.

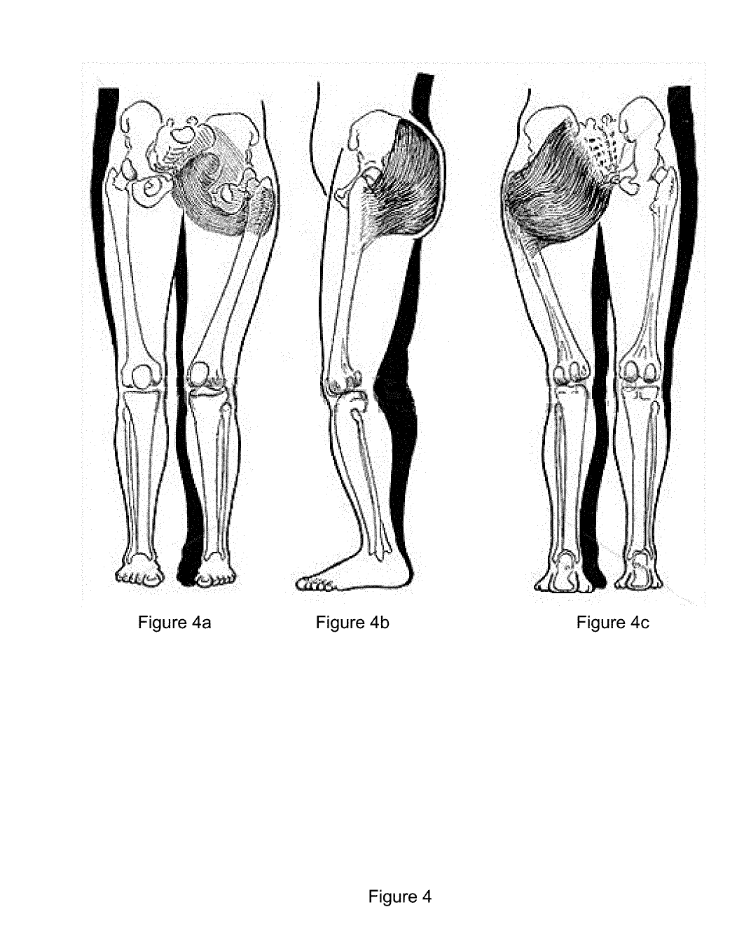

Specifically, referring to FIG. 4, there is shown the contrasted effects of weakened gluteus maximus muscles. Specifically, FIGS. 4a and 4b shows the upper outward rotation and lateral sway of the hip and therefore the lower inward rotation of the knee and the rolling of the foot. Specifically, FIG. 4b shows the exacerbated tilt of the pelvis.

The most important role of the gluteus medius is to stabilize the hip, pelvis, and femur, in the frontal plane. When standing on a single leg (for example the stance phase of gait) it is the responsibility of the gluteus medius to maintain a relatively level pelvis. This function plays a significant role in maintaining optimal length/tension of the muscles of the core, and arthrokinematics of the hip, SIJ, and lumbar facets. Gluteus medius weakness and inhibition results in a drop of the opposite side of the pelvis during single leg stance, referred to as a “positive Trendelenburg sign.”

The gluteus medius also maintains femoral alignment ensuring optimal arthrokinematic of the knee. The optimal alignment of the femur ensures better congruence between the femoral condyles and tibial plateau. This congruence is important for spreading load evenly through intra-articular tissues. The dysfunctional pattern noted in individuals with a weak or inhibited gluteus medius has high level of congruence with common wear patterns and ligament damage seen in common knee impairments.

By maintaining optimal alignment of the femur and pelvis the gluteus medius maintains optimal length/tension of hip musculature. Further, this maintains the oblique axis of the gluteus maximus muscle fibers, resulting in optimal production from force couples during hip extension.

As such, decrease gluteal activation contributes to a number of biomechanical problems including:
a. Hamstring strains: Due to delayed gluteus maximus activity, the hamstring muscles become dominant during hip extension, which causes hamstring strains. Indeed, many athletes suffer repetitive hamstring injuries on account of the incorrect remedial focus of strengthening the hamstrings whereas strengthening of the gluteus maximus would be more appropriate;
b. Lower back pain: Gluteus maximus activation plays an important role in stabilising the pelvis during the task of lifting. Delayed gluteus maximus activation causes excessive compensation of the back extensors;
c. Anterior knee pain: The excessive internal rotation of the femur as a result of gluteus medius weakness increases the pressure on the patellar cartilage;
d. Anterior hip pain: Decreased force production from the gluteus maximus during hip extension is associated with increased anterior translation of the femur in the acetabulum. The increased femoral anterior glide leads to increased force and wear and tear on the anterior hip joint structures;
e. Lower-body mal-alignment: Weak gluteals results in increased internal rotation of the femur, knee valgus and foot pronation;
f. Anterior cruciate ligament (ACL) sprains;
g. Chronic ankle instability; and
h. Iliotibial friction syndrome.

FIGS. 5-7 show and orthotic 100 comprising strapping 105 configured for muscle imbalance correction, especially the commonplace gluteus maximus and medius muscle imbalance correction.

So as to provide muscle imbalance correction, the strapping 105 is configured for pulling substantially in-line with muscle fibres of an undercompensating muscle group (these typically being the weakened muscle group) to assist activation of the undercompensating muscle group and compressing across an overcompensating muscle group (these typically being the muscles overcompensating for the weakened muscles) to provide resistance for the overcompensating muscle group.

In this manner, the strapping 105 advantageously enhances and promotes the activation of the weakened muscles while inhibiting or resisting those overcompensating muscles.

For example, for the above-mentioned muscle imbalance syndrome which may be caused by a sedentary lifestyle, the gluteus maximus, medius and minimus, vastus medialis and vastus medialis oblique may become weakened, whereas the tensor fascia latae, iliopsoas become strengthened and/or shortened through overcompensation.

As such, in a preferred embodiment, strapping 105 is adapted to pull substantially in-line with an undercompensating muscle group comprising at least one of the gluteus maximus, medius and minimus and the vastus medialis and vastus medialis oblique. By pulling in line with these muscles, the activation of these muscles is favoured allowing for the subsequent strengthening through shortening the length of pull of the muscle.

Furthermore, in this preferred embodiment, the strapping 105 is adapted to compress across the muscle fibres of an overcompensating muscle group comprising at least one of the tensor fascia latae and iliopsoas. As such, by compressing along these muscles, these muscles are resisted and their activation becomes disfavoured.

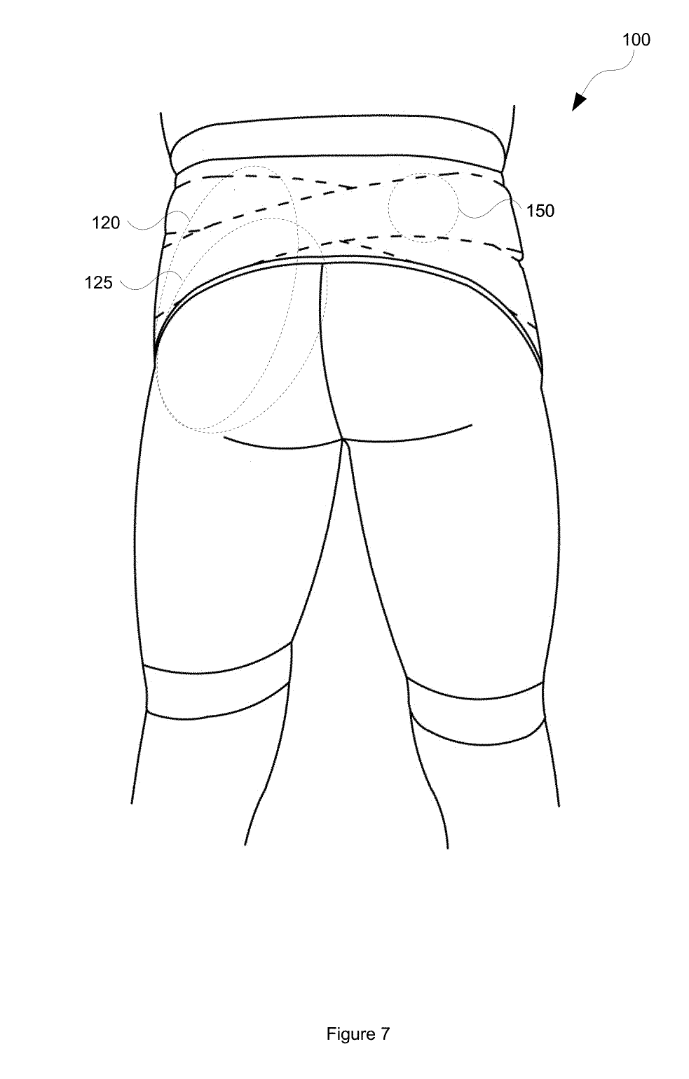

Specifically, referring to FIG. 7, there is shown the approximate locations of the gluteus maximus 125 and gluteus medius 120 muscles.

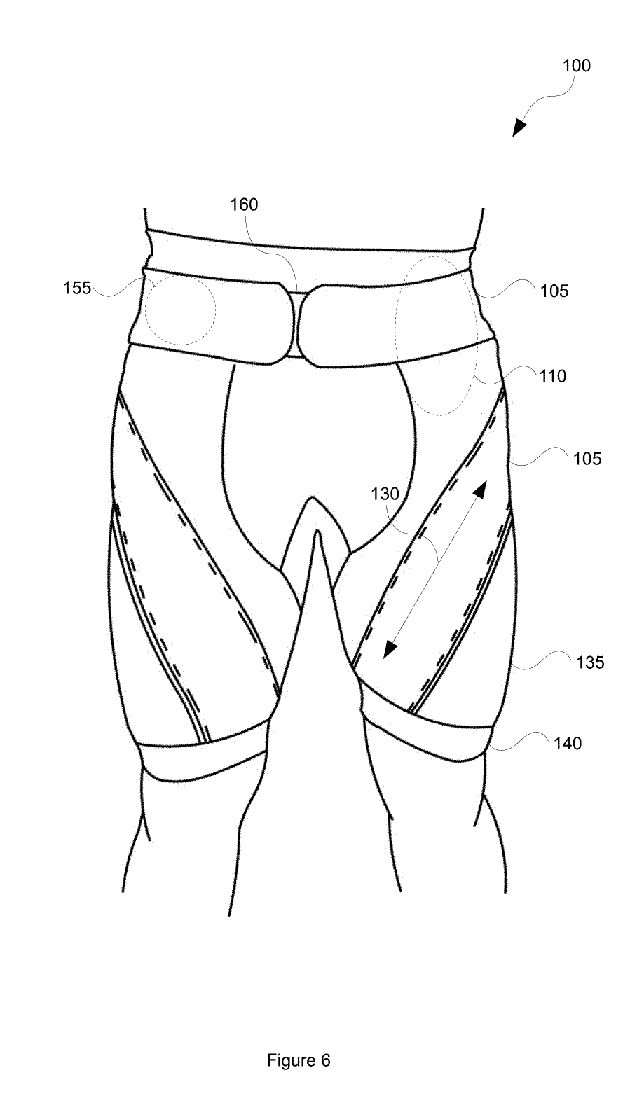

Furthermore, referring to FIGS. 5 and 6, there is shown the approximate locations of the vastus lateralis 115 and tensor fascia latae 110 muscles.

As is apparent, the strapping 105 is adapted to pull along force vector 130 thereby substantially pulling in line with the muscle fibres of the gluteus maximus 125, gluteus medius 120, gluteus minimus (not shown) and the vastus lateralis 115 so as to favour the activation of these muscles. Furthermore, the strapping 105 is adapted to compress against (i.e. to pull across the muscle fibres of) the tensor fascia latae 110 and the iliopsoas (not shown) so as to disfavour the activation of these muscles.

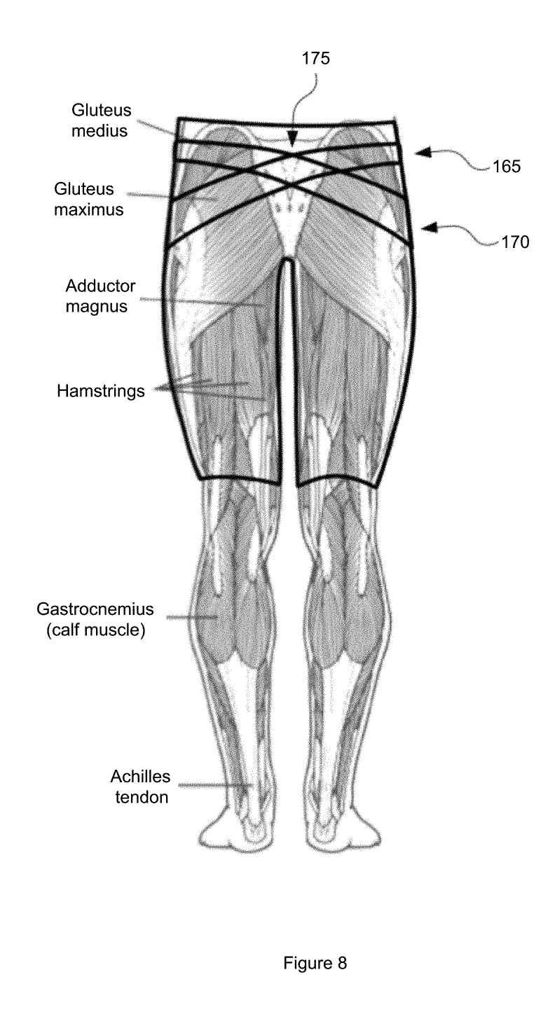

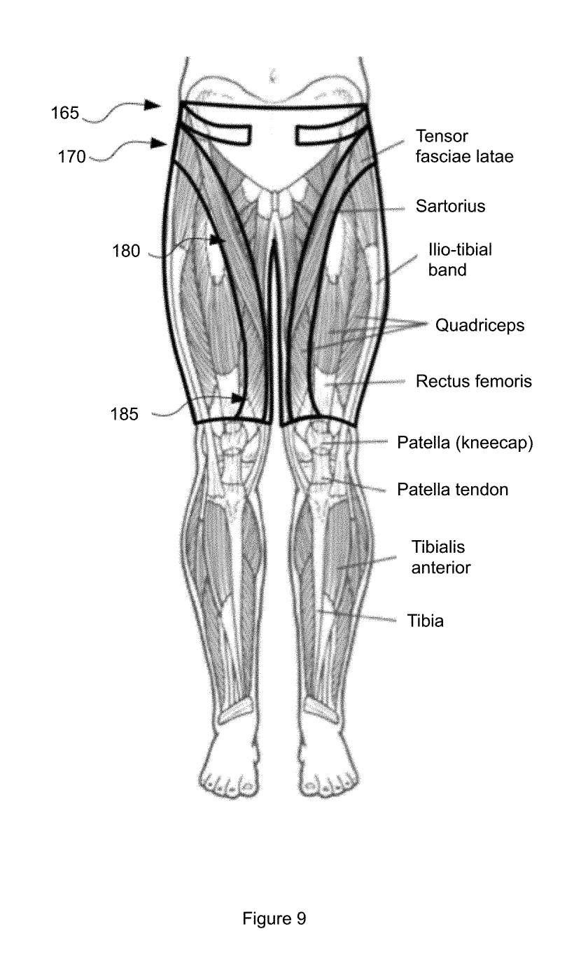

Turning now to FIG. 8 there is shown a posterior view of the orthotic 100 overlaid various muscles. Similarly, FIG. 9 shows an anterior view of the orthotic 100 overlaid the muscles.

Specifically, referring to FIG. 8, as can be seen, the strapping 105 comprises a pair of straps. Each strap travels respectively posteriorly from respective opposite superior hip regions 165 across a pelvic region to respective opposite inferior hip regions 170.

As can be seen, the straps, travelling in this way, physically resist the lateral overcompensating muscle group at the superior hip region 165 and the inferior hip region 170. In other words, the straps, by travelling around and physically compressing at these regions resists and therefore deactivates the overcompensating muscle group (comprising at least one of the tensor fascia latae and iliopsoas) so as to deactivate this overcompensating muscle group.

Furthermore, the straps, by travelling downwards across the pelvis region 175 are able to physically pull substantially in line with the undercompensating muscle group (comprising at least the gluteus maximus, medius, and minimus, vastus medialis and vastus medialis oblique) so as to physically favour the activation of these muscles.

As can be appreciated, the orthotic 100, by favouring the activation and therefore strengthening of the gluteus 120, 125 and vastus medialis muscles 115 and by disfavouring the activation and therefore weakening of the tensor fascia latae 110 and iliopsoas, allows for the correction of the muscle imbalance.

It should be noted that the strapping 105 is adapted to physically pull along the force vector 130 as opposed to merely compress the region for proprioceptive input as the abovementioned prior art does. In this manner, the strapping 105 is preferably physically reinforced so as to suitable for physically pulling in line and across muscle groups as is described herein.

Furthermore, to increase the compression of the overcompensating muscle group, the tensor fascia latae 110 and the iliopsoas in this embodiment, the orthotic 110 may comprise padding adapted to sit between the inner surface of the strapping 105 and this muscle group to increase the compression force and therefore the force applied across the muscle fibres to disfavour the activation of this muscle group.

In embodiments, the straps 105 morphs around the anatomy of the leg by starting at the inner knee following the muscle fibres of vastus medialis oblique, then along the line of the Sartorius, then cut perpendicular across tensor fascia latae to inhibit it. As the strap further wraps around the back on it follows the muscle fibre lines of gluteal muscles wherein, as the strap comes across to the opposite side the strap thins out to compress the tensor fascia latae again whilst remaining thin at the front of the hip joint so not to affect any comfort or range of movement.

### Posture Correction

In a preferred embodiment, the orthotic 100 is further configured for posture correction. Specifically, referring again to FIG. 4, FIGS. 4a and 4c shown the hip exhibiting and inwards rotation on account of the weakened gluteus maximus muscle.

Now, referring to at least FIGS. 5 and 6, it is appreciated that the strapping 105, by travelling across and physically pulling across the anterior thigh surface from the outer thigh to the inner thigh, is adapted to physically exert a rotational force on the thigh allowing for the lateral rotation of the hip to a substantially neutral position.

Specifically, referring to FIG. 9, as can be seen, each strap travels from the inferior hip region 170 across a thigh surface to an inner thigh, medial knee region 185 to as to be able to physically impart the rotational forces on the legs.

Furthermore, FIG. 4b shows the excessive tilt of the pelvis. The tilting of the pelvis is caused by the weakening of the gluteus maxim us muscle which can no longer maintain the correct poise of the pelvis. In this manner, as is shown in the figure, the pelvis exhibits a tilt.

Referring again to at least FIGS. 5 and 6, it is appreciated that the strap 105 exhibits not only a rotational force as described above, but also an extension force for extending the hip to favour a substantially neutral pelvis tilt. In this regard, it should be noted that in a preferred embodiment, the strapping terminates substantially at the inner knee region of the patient so as to be able to adequately provide this extension force. Were the strapping 105 to terminate at a higher position upward of the knee, the strapping 105 would not necessarily be able to deliver the same magnitude of extension force adapted to return the hip to a substantially neutral pelvis tilt.

### Lumbopelvic Support

In a preferred embodiment, in addition to the orthotic 100 providing the above-mentioned muscle imbalance and posture correction, the orthotic 100 is adapted to provide lumbopelvic stability.

The provision of lumbopelvic stability often times decreases pain by providing adequate support therefore allowing for the subsequent muscle balancing and posture correction exercises.

Now, by way of background, the pelvis is large bone consisting of two large semi circles articulating at two joints; the pubic symphysis and the sacroiliac joints.

These joints are configured for stability and stress reduction when transferring weight and force from the lower limbs to the lower lumbar spine. The pelvis and the sacroiliac joints comprises significant ligamental support for stability, known as form closure. Furthermore, stability is further provided by the horizontal fibres of the gluteus maximus known as force closure

Form closure is compromised through excessive wear from certain activity and/or lack of dampening and strength from the force closure muscles, trauma and pregnancy. Furthermore, form closure is compromised through muscle imbalances from repetitive tasks and sedentary lifestyles.

Compromised form closure increases shear forces acting on the lumbar spine when load bearing and weight transfer.

As such, in a preferred embodiment, the strapping 105 is further configured for lumbopelvic support. In this manner, the strapping 105 is configured for physically compressing the pelvis to enhance form closure. Specifically, the strapping 105 is adapted to compress the anterior superior iliac spine (ASIS) and the posterior superior iliac spine (PSIS) skeletal landmarks.

Specifically, referring to FIG. 6, there is shown the approximate location of the ASIS 155 and referring to FIG. 7, there is shown the approximate location of the PSIS 150.

In embodiments, and so as to enhance the compression of the ASIS 155 and the PSIS 150, the orthotic 100 may comprise appropriately located cushioning, padding or the like, such as by way of strategically placed memory foam pads adapted to sit between the inner surface of the strapping 105 and the ASIS 155 and PSIS 150 to enhance the compressive force at these regions.

### Compression

In a further preferred embodiment, the orthotic 100 comprises a compression garment 135.

The compression garment 135 provides compression to enhance recovery.

In a preferred embodiment, the compression garment 135 comprises a sleeve or the like within which the strapping 105 is slidably retained.

### Upper Body Orthotic

It should be noted that whereas the orthotic 100 has been described with reference to the preferred embodiment comprising the orthotic been suited for lower body utilisation, in embodiments, the orthotic 100 may be utilised additionally for upperbody muscle imbalance correction.

Specifically, referring to FIGS. 10 and 11, there is shown an orthotic 190 for upperbody use.

Specifically, likewise for the lower body, upper cross syndrome is responsible for up to 80% of chronic cases which include headaches forward head posture, shoulder impingement, cervical disc degeneration, facet joint degeneration. The primary reason is that humans do most things in front and often build up strength in pectoralis muscles and anterior deltoids which pull the whole shoulder complex forwards.

As a result shoulder and back stabilisers become stretched. This upper body orthotic aims to correct this by shortening and pulling in line with the fibres to reactivate this muscles as well as pulling across and against the pectoralis and deltoids muscles.

As such, in the embodiments shown the straps of the upperbody orthotic 190 starts at the front of the shoulder and pull across pectorals major and anterior deltoid to inhibit these muscles.

The upperbody orthotic 190 also pulls in the opposite direct of pectoralis minor to inhibit this muscle.

The straps of the the upperbody orthotic 190 then follows around the shoulder and pulls in line with posterior deltoid and posterior rotator cuff muscles, namely infraspinatus.

The straps then pulls in line with shoulder stabilisers such as lower trapezius and then, to facilitate serrated anterior, the strapping wraps around the ribs towards the front.

As such, the upperbody orthotic 190 addresses the upper body cross dysfunction wherein tightness of pectoralis muscles forces the shoulder forced to elevate instead of pivoting thus putting pressure on the neck with the use of neck muscles to elevate the shoulder.

### Construction

In a preferred embodiment, the orthotic 100 takes the form of the short leggings as are substantially provided in the figures so as to encompass substantially from the pelvis region of the patient to the knee region. However, should be appreciated that in other embodiments, the orthotic 100 could take the form of long leggings and the like.

As alluded to above, in a preferred embodiment, the orthotic 100 comprises a compression garment 135 to enhance recovery.

Furthermore, the orthotic 100 comprises the strapping 105 adapted to provide the above-mentioned muscle imbalance correction, posture correction and lumbopelvic support. In a preferred embodiment, the compressive material 135 comprises elongate sleeving within which the strapping 105 is slidably engaged. However, it should be noted that in embodiments, the strapping 105 may be located at an external or interior surface of the compressive material 135.

In a preferred embodiment, the strapping 105 is substantially elasticised so as to be able to provide the pulling force and torque necessary to achieve the above-mentioned muscle imbalance and posture correction and lumbopelvic support.

In one embodiment, the strapping 105 has a width of substantially 10 cm.

As can be appreciated from the figures, the orthotic 100 comprises a thigh strapping 105 portion for each leg and in this regard, the strapping 105 comprises a thigh portion adapted to travel across the anterior thigh surface from an upper lateral thigh to a lower medial thigh sides. In this manner, the thigh strapping portion is able to provide the muscle imbalance correction through the activation and resistance of the above-mentioned respective weakened and overcompensating muscle groups, and provide the posture correction through the exertion of lateral hip rotation and hip extension forces.

As alluded to above, the strapping 105 of the thigh portion travels to substantially a knee region of the patient so as to especially provide the hip extension forces necessary. Furthermore, the thigh portion strapping 105 may terminate at a knee region engagement 140 wherein, in a preferred embodiment, the knee region engagement 140 comprises a frictional engagement adapted to fractionally engage the skin at the knee region of the patient. In one embodiment, the frictional engagement may comprise an inwardly orientated rubberised surface or the like adapted to engage the skin of the patient. In this embodiment, the remainder of the thigh region portion of the strapping 105 may slide with respect to the skin of the patient.

Furthermore, the strapping 105 comprises a pelvis portion adapted to substantially compress the ASIS 155 and PSIS 150 of the pelvis so as to provide the lumbopelvic support.

Referring to FIG. 7, in a preferred embodiment, the strapping 105 is continuous so as to travel from the thigh portion to the pelvis portion as a continuous strap. As can be seen from FIG. 7, the strapping 105 from each respective leg crosses over substantially at the coccyx of the patient.

Furthermore, in a preferred embodiment, the strapping 105 is length and therefore tension adjustable.

Specifically, referring to FIG. 6, there is shown the strapping 105 comprising a length adjusting portion 160. In this embodiment, the length adjusting portion 160 may comprise Velcro′ fasteners adapted to allow the user to control the length of the strapping 105.

In this manner, by adjusting the length adjusting portion 160, the patient may control the tension of the strapping 105 all the way down to the thigh portions of the strapping 105. In this manner, in use, the patient may advantageously initially utilise the orthotic 105 with strapping 105 at high tension wherein, as recovery progresses, the strapping 105 may be loosened.

### Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the orthotic industries.

1: An orthotic configured for muscle imbalance correction wherein the orthotic comprises strapping configured for:
physically activating an undercompensating muscle group comprising at least one of the gluteus maximus, medius, and minimus, vastus medialis and vastus medialis oblique by physically pulling substantially in-line with muscle fibres of the undercompensating muscle group to assist muscle activation; and
physically deactivating an overcompensating muscle group comprising at least one of the tensor fascia latae and iliopsoas by physically pulling and therefore compressing across muscle fibres of the overcompensating muscle group to provide muscle resistance.

2: The orthotic as claimed in claim 1, wherein the strapping comprises a pair of straps configured for respectively travelling posteriorly from respective opposite superior hip regions across a pelvic region to respective opposite inferior hip regions so as to be configured for:
physically pulling around the overcompensating muscle group at the superior and inferior hip regions so as to physically deactivate the overcompensating muscle group at these regions; and
physically pulling in line with the undercompensating muscle group between the at the superior and inferior hip regions so as to physically activate the undercompensating muscle group.

3: The orthotic as claimed in claim 1, wherein the straps are tension adjustable.

4: The An orthotic as claimed in claim 3, wherein the orthotic comprises ergonomically located lateral anterior strap tension adjustment means positioned to allow the wearer to self-adjust the tension of the straps.

5: The orthotic as claimed in claim 2, wherein the orthotic is further configured for posture correction wherein the strapping is further configured for physically exerting hip rotation forces for the lateral rotation of the hips to favour a substantially neutral hip position.

6: The orthotic as claimed in claim 5, wherein the pair of straps are configured for travelling from the respective opposite inferior hip regions across respective anterior thigh surfaces to exert lateral rotational forces on each leg.

7: The orthotic as claimed in claim 6, wherein the pair of straps each terminate at respective medial knee regions

8: The orthotic as claimed in claim 8, wherein the orthotic comprises a frictional engagement to enhance the purchase of the straps at at least one of respective thigh surfaces and medial knee regions.

9: The orthotic as claimed in claim 5, wherein the strapping is further configured for physically exerting hip extension forces for the extension of the hip to favour a substantially neutral pelvis tilt.

10: The orthotic as claimed in claim 2, wherein the orthotic is further configured for lumbopelvic support wherein the strapping is configured for physically exerting compressive forces for compressing the pelvis to enhance form closure.

11: The orthotic as claimed in claim 11, wherein the strapping is configured for compressing the anterior superior iliac spine (ASIS) and posterior superior iliac spine (PSIS).

12: The orthotic as claimed in claim 12, wherein the orthotic further comprises appropriately padding located substantially at at least one of the ASIS and PSIS regions to enhance compressive force exerted by the straps.

13: The orthotic as claimed in claim 2, further comprising a compression garment.

14: The orthotic as claimed in claim 14, wherein the compression garment comprises a sleeve for slideably engaging at least a portion of the straps.
